SEQUENCE LISTING (14 sequences)

SEQ ID NO: 1 - DNA, 20 bp, Mus musculus
actggccagatccactcatg

SEQ ID NO: 2 - DNA, 21 bp, Mus musculus
gaacggcaggcagatgatcag

SEQ ID NO: 3 - DNA, 31 bp, Rattus rattus
taggatccaccatgagcaagcagaaccactt

SEQ ID NO: 4 - DNA, 20 bp, Rattus rattus
acatgaacagcatgctggac

SEQ ID NO: 5 - DNA, 24 bp, Rattus rattus
gattctccgaatctagaagttcca

SEQ ID NO: 6 - DNA, 28 bp, Rattus rattus
tactcgagttaacgagatgtggaagcca

SEQ ID NO: 7 - DNA, 1083 bp, Mus musculus
atgagcaagtcagaccattttctagtgataaacggcaagaactgctgtgtgttccgagatgaaaacatcgccaaggtcttgccaccggtgttggggctggaatttgtgttcggactcctgggcaatggccttgccttgtggattttctgtttccacctcaagtcctggaaatccagccggattttcttgttcaacttggccgtggctgactttctcctgatcatctgcctgccgttcctgacggacaactatgtccataactgggactggaggttcggaggcatcccttgccgtgtgatgctcttcatgttggctatgaaccgacagggcagcatcatcttcctcaccgtggtggctgtggaccgctacttccgggtggtccatccacaccacttcttgaacaagatctccaaccggacggcggccatcatttcttgcttcttgtggggtctcaccatcggcctgactgtccacctcctctatacaaacatgatgaccaaaaatggcgaggcatatctgtgtagcagcttcagcatctgttacaacttcaggtggcacgatgctatgttcctcttggaattcttcttgcccctggccatcatcttgttctgctcaggcaggatcatctggagcctgaggcagagacagatggacagacatgccaagatcaagagggccatcaacttcatcatggtggtggctattgtattcatcatttgcttcctacccagtgtggctgtgcgcatccgcatcttctggcttctctacaaatataacgtacgcaactgtgacatctactcctcggtggacctggctttctttaccacccttagctttacctacatgaacagcatgctggaccctgtggtctactatttctccagcccatctttccccaacttcttctccacgtgtatcaaccgctgccttcgaaagaaaacattgggtgaacccgataataaccgaagcactagtgtggagctcacgggggaccccagcacaaccagaagtattccaggggcgctaatggctgaccccagtgagccaggcagccccccttatctggcttccacatctcgttaa

SEQ ID NO: 8 - protein, 360 aa, Mus musculus
MSKSDHFLVINGKNCCVFRDENIAKVLPPVLGLEFVFGLLGNGLALWIFCFHLKSWKSSRIFLFNLAVADFLLIICLPFLTDNYVHNWDWRFGGIPCRVMLFMLAMNRQGSIIFLTVVAVDRYFRVVHPHHFLNKISNRTAAIISCFLWGLTIGLTVHLLYTNMMTKNGEAYLCSSFSICYNFRWHDAMFLLEFFLPLAIILFCSGRIIWSLRQRQMDRHAKIKRAINFIMVVAIVFIICFLPSVAVRIRIFWLLYKYNVRNCDIYSSVDLAFFTTLSFTYMNSMLDPVVYYFSSPSFPNFFSTCINRCLRKKTLGEPDNNRSTSVELTGDPSTTRSIPGALMADPSEPGSPPYLASTSR

SEQ ID NO: 9 - DNA, 1083 bp, Rattus rattus
atgagcaagcagaaccactttctggtgataaacggcaagaactgctgtgtgttccgagatgaaaacatcgccaaggtcctgccgccggtgttggggctggagtttgtgtttggactcctgggtaatggccttgccttgtggatcttctgtttccatctcaaatcctggaaatccagccggattttcttgttcaacctggccgtggctgactttctcctgatcatttgcttgccgttcttgacggacaactatgtccagaactgggactggaggttcgggagcatcccctgccgcgtgatgctcttcatgttggccatgaaccgacagggcagcatcatcttcctcacggtggtggctgtggacaggtacttcagggtggtccacccgcaccacttcctgaacaagatctccaaccggacggcggccatcatctcttgcttcctgtggggcatcaccatcggcctgacagtccacctcctctacacggacatgatgacccgaaacggcgatgcaaacctgtgcagcagttttagcatctgctacactttcaggtggcacgatgcaatgttcctcttggaattcttcctgcccctgggcatcatcctgttctgctctggcaggatcatttggagcctaaggcagagacagatggacaggcacgtcaagatcaagagggccatcaacttcatcatggtggttgccattgtgtttgtcatctgcttcctgcccagtgtggccgtgaggatccgcatcttctggctcctctacaaacacaacgtgaggaactgtgacatctactcctctgtggacttggccttcttcaccacccttagctttacctacatgaacagcatgctcgacccggtggtctactatttctccagcccatctttccccaacttcttctccacgtgcatcaaccgttgccttcgaaggaaaaccttgggcgaaccagataataaccggagcacgagtgtggagctcacgggggaccccagcacaatcagaagtattccaggggcattaatgactgaccccagtgagccaggcagccccccttatctggcttccacatctcgttaa

SEQ ID NO: 10 - protein, 360 aa, Rattus rattus
MSKQNHFLVINGKNCCVFRDENIAKVLPPVLGLEFVFGLLGNGLALWIFCFHLKSWKSSRIFLFNLAVADFLLIICLPFLTDNYVQNWDWRFGSIPCRVMLFMLAMNRQGSIIFLTVVAVDRYFRVVHPHHFLNKISNRTAAIISCFLWGITIGLTVHLLYTDMMTRNGDANLCSSFSICYTFRWHDAMFLLEFFLPLGIILFCSGRIIWSLRQRQMDRHVKIKRAINFIMVVAIVFVICFLPSVAVRIRIFWLLYKHNVRNCDIYSSVDLAFFTTLSFTYMNSMLDPVVYYFSSPSFPNFFSTCINRCLRRKTLGEPDNNRSTSVELTGDPSTIRSIPGALMTDPSEPGSPPYLASTSR

SEQ ID NO: 11 - DNA, 1092 bp, Monkey
atgaatcggcaccatctgcaggatcactttctggtaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctcctgatcatctgcctgccattcctgatggacaactatgtgagacgttgggactggaagtttggggacatcccttgccggctgatgctcttcatgctggccatgaaccgccagggcagcatcatcttcctcacggtggtggccgtggacaggtatttccgggttgtccatccccaccacgccctgaacaagatctccaatcgaacagcagccatcatctcttgccttctgtggggtgtcactattggcctgacagtccacctcctgaagaggaagatgccgatccagaatggcactgcgaatctgtgcagcagcttcagcatctgcaataccttccggtggcacgaagccatgttcctcctggagttcttcctgcccctgggcatcatcctgttctgctcagccagaattatctggagcctgcggcagagacaaatggaccggcatgccaagatcaaaagagccatcaccttcatcatggtcgtggccatcgtctttgtcatctgcttccttcccagcgtggctgtgcggatacgcatcttctggctcctgcacacttttggcacgcagaactgtgaagtgtaccgctcggtggacctggcgttctttatcactctcagtttcacctacatgaacagcatgctggaccccgtggtgtactacttctccagcccatcctttcccaacttcttctccactttgatcaaccgctgcctccgaaggaagatgacaggtgacccagataataaccgcagcacgagtgtcgagctcacgggggacccgaacacaaccagaggcgctccagaggcattaatggccaaccccagtgagccatggagcccctcttatctgggtccaacctctcgttaa

SEQ ID NO: 12 - protein, 363 aa, Monkey
MNRHHLQDHFLVIDKKNCCVFRDDFIVKVLPPVLGLEFIFGLLGNGLALWIFCFHLKSWKSSRIFLFNLAVADFLLIICLPFLMDNYVRRWDWKFGDIPCRLMLFMLAMNRQGSIIFLTVVAVDRYFRVVHPHHALNKISNRTAAIISCLLWGVTIGLTVHLLKRKMPIQNGTANLCSSFSICNTFRWHEAMFLLEFFLPLGIILFCSARIIWSLRQRQMDRHAKIKRAITFIMVVAIVFVICFLPSVAVRIRIFWLLHTFGTQNCEVYRSVDLAFFITLSFTYMNSMLDPVVYYFSSPSFPNFFSTLINRCLRRKMTGDPDNNRSTSVELTGDPNTTRGAPEALMANPSEPWSPSYLGPTSR

SEQ ID NO: 13 - DNA, 1254 bp, Rabbit
gctagttaagcttggtaccgagctcggatccactagtccagtgtggtggaattgccctttgcgcggccccagtctggtctccccatgaaccagcaccgcccgcagagccattttctggagatagacaagaagaactgctgcgtgttccgagatgacttcatcgccaacgtgctgccgcccgtgctggggctggagttcgtgttcgggctgctgggcaatggcctcgccttgtggattttctgcttccacctcaagtcctggaaatccagccggattttcctgttcaacctggccgtggctgacttcctcctgatcatttgcctgcccttcctgacggacaactatatgaggaagtgggattggaggttcggggacatcccatgccggctgatgctcttcatgctggccatgaaccgccagggcagcatcatcttcctcacggtggtcgccgtggacaggtacttccgggtggtccaccctcaccacgccctgaacaagatctccaaccggacagcggccgccatctcctgcctgctgtggggcgtcaccatcggcctgacggtccacctcctgcgcaaaaggatgctgacccagaacggcccggccaatctgtgcagcagcttcagcatctgcaacaccttccggtggcacgacgccatgttccttctggagttcttcctgcccctcgccatcatcctcttctgctccgtccggatcgtctggagcctgcggcagaggcagatggacaggcacgtcaagatcaagagggccatcaacttcatcatggtggtggccgtggtcttcatcatctgcttcctgcccagcgtggccgtgcggatgcgtatcttctggctcctgcgcacggcggggacgcaggactgtgacgtgtaccgctccgtcgacctggccttcttcatcaccctcagcttcacctacatgaacagcatgctggaccccctggtctactacttctccagcccctcgttccccaacttcttctccgcgctcatcaaccgctgcctgcggaggagcccagcaggtgagccggagaacaacaggagcaccagcgtggagctcactggggacccgagcaccgctcggagcgctccggacgcgctagtggccgagcccaacgggccacggagcccctcctacctggtcccaaatcctcgttagacggtggttccgaggaaggagactgtcgcaaagggcaattctgcagatatccagcacagtggcggccgctcgagtcta

SEQ ID NO: 14 - protein, 363 aa, Rabbit
MNQHRPQSHFLEIDKKNCCVFRDDFIANVLPPVLGLEFVFGLLGNGLALWIFCFHLKSWKSSRIFLFNLAVADFLLIICLPFLTDNYMRKWDWRFGDIPCRLMLFMLAMNRQGSIIFLTVVAVDRYFRVVHPHHALNKISNRTAAAISCLLWGVTIGLTVHLLRKRMLTQNGPANLCSSFSICNTFRWHDAMFLLEFFLPLAIILFCSVRIVWSLRQRQMDRHVKIKRAINFIMVVAVVFIICFLPSVAVRMRIFWLLRTAGTQDCDVYRSVDLAFFITLSFTYMNSMLDPLVYYFSSPSFPNFFSALINRCLRRSPAGEPENNRSTSVELTGDPSTARSAPDALVAEPNGPRSPSYLVPNPR

(19) United States
(12) Patent Application Publication
(10) Pub. No.: US 2004/0254224 A1
(43) Pub. Date: Dec. 16, 2004

(54) Medicaments

(76) Inventors: Steven Michael Foord, Stevenage (GB); Nicholas Brian Pike, Stevenage (GB); Alan Wise, Stevenage (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/484,788

(22) PCT Filed: Apr. 10, 2002
(86) PCT No.: PCT/GB02/01671

(30) Foreign Application Priority Data
Apr. 11, 2001 (GB) 0109028.1
Nov. 6, 2001 (GB) 0126637.8

(51) Int. Cl. A61K 31/455

(52) U.S. Cl. 514/355

(57) ABSTRACT

A method of treating HM74A and/or HM74 mediated disease in an individual which comprises administering to the individual a therapeutic amount of a modulator of HM74A and/or HM74 activity.

### FIELD OF THE INVENTION

This invention relates to the identification of the closely related orphan G-protein coupled receptors HM74A and HM74 as high and low affinity receptors, respectively, for the ligand nicotinic acid and their use in screening methods, and rational drug design, to identify modulators of these receptors, to the modulators so identified and their use in therapy. The present invention also relates to a method of treatment of diseases mediated by HM74 and HM74A comprising the administration of a therapeutically effective amount of a modulator of the receptors HM74 and HM74A.

### BACKGROUND OF THE INVENTION

G-protein coupled receptors (GPCRs), otherwise known as 7TM receptors are a super-family of membrane receptors that mediate a wide variety of biological functions. Upon binding of extracellular ligands, GPCRs interact with a specific subset of heterotrimeric G proteins that can, in their activated forms, inhibit or activate various effector enzymes and/or ion channels. All GPCRs are predicted to share a common molecular architecture consisting of seven transmembrane helices linked by alternating intracellular and extracellular loops. The extracellular receptor surface has been shown to be involved in ligand binding whereas the intracellular portions are involved in G protein recognition and activation. Different G-protein c-subunits preferentially stimulate particular effectors to modulate various biological functions in a cell. G-protein coupled receptors are found at numerous sites within a mammalian host. Over the past 15 years many therapeutic agents targetting 7TM receptors have been successfully introduced onto the market, thereby establishing their value as therapeutic targets.

Activation of receptors coupled to the Gᵢ family of G proteins leads to inhibition of adenylate cyclase and lowering of intracellular cAMP levels. In adipose tissue a reduction of cAMP levels results in an inhibition of hormone-sensitive lipase (HSL) activity, an enzyme which regulates the process of lipolysis (i.e. the hydrolysis of triglycerides (TG) to glycerol and non-esterified fatty acids (NEFA)).

Inhibition of adipocyte lipolysis resulting in a reduction of plasma NEFA levels is thought to reduce hepatic triglyceride synthesis resulting in a decreased output of TG-rich lipoproteins (VLDL). A reduction in VLDL synthesis would then produce a functional inhibition of cholesterol ester transfer protein (CETP) activity resulting in an elevation in high-density lipoprotein (HDL) levels. This alteration in lipoprotein levels, decreased TG and increased HDL, would be suitable for the treatment of dyslipidernic patients and should result in a decreased risk of cardiovascular disease. Furthermore, inhibition of adipocyte lipolysis via the activation of an, as yet, undefined Gi-coupled receptor is thought to be an important mechanism of action of nicotinic acid (Niacin). Nicotinic acid has been used clinically for over 40 years in patients with various forms of hyperlipoproteinaemia. Nicotinic acid produces a very desirable alteration in lipoprotein profiles; reducing levels of VLDL, LDL and Lp(a) whilst increasing HDL. Nicotinic acid has also been demonstrated to have disease modifying benefits, reducing the progression and increasing the regression of atherosclerotic lesions and reducing the number of cardiovascular events in several trials.

### SUMMARY OF THE INVENTION

The present invention is based on the finding that the G-protein coupled receptors HM74A and HM74 are able to act as receptors for nicotinic acid and that cells transfected to express HM74A and/or HM74 gain the ability to elicit Gᵢ G protein mediated responses following exposure to nicotinic acid. Identification of a ligand for HM74A and/or HM74 therefore facilitates the development of screening methods for identifying modulators of the receptor.

Accordingly the invention further provides a method for identifying agents which modulate the activity of the HM74A and/or HM74 receptor, which comprises determining whether the test agent interacts with HM74A and/or HM74. The method may comprise the use of the HM74A and/or HM74 receptor in combination with a ligand therefore, e.g. nicotinic acid.

The invention further comprises the use of agents identified using the method of the invention in the treatment of diseases mediated by HM74 and/or HM74A and their use in the manufacture of a medicament for the treatment of HM74 and/or HM74A mediated diseases.

Accordingly, the invention further provides a method of treatment of diseases or conditions mediated by HM74A and/or HM74 in an individual which comprises the administration of a therapeutically effective amount of an HM74A and/or HM74 receptor modulator. The present invention excludes by specific proviso the use of nicotinic acid in the method of the invention. The invention also provides the use of a modulator of HM74A and/or HM74 in the manufacture of a medicament for the treatment of diseases or conditions mediated by HM74A or HM74 with the proviso the modulator is not nicotinic acid.

### BRIEF DESCRIPTION OF THE FIGURES

FIG. 1. Co-expression of HM74, but not of other orphan receptors, with Gₒ₁α in HEK293T cells results in nicotinic acid-mediated stimulation of [³⁵S]GTPγS binding

FIG. 2. Taqman mRNA analysis demonstrate distribution of HM74/HM74A in adipose, omentum and spleen.

FIG. 3. Nicotinic acid stimulates [³⁵S]GTPγS binding in membranes from HEK293T cells co-expressing HM74 and Gₒ₁α in a concentration-dependent manner

FIG. 4. Nicotinic acid, but not its metabolite nicotinuric acid, can stimulate [³⁵S]GTPγS binding in membranes from HEK293T cells co-expressing HM74 and Gₒ₁α.

FIG. 5. Nicotinic acid-mediated stimulation of [³⁵S]GTPγS binding in membranes from HM74-expressing HEK293T cells is sensitive to pertussis toxin pretreatment

FIG. 6. HM74 couples equally well to the Gᵢ-like G proteins Gᵢ₁α, Gᵢ₂α, Gᵢ₃α and Gₒ₁α following co-expression in HEK293T cells

FIG. 7. Nucleotide sequence of murine HM74A

FIG. 8. Amino acid sequence of murine HM74A

FIG. 9. Alignment of murine HM74A and human HM74 protein sequences

FIG. 10. cDNA sequence of rat HM74A

FIG. 11. Amino acid sequence of rat HM74A

FIG. 12. Amino sequence alignment of murine and rat HM74A

FIG. 13. Alignment of rat HM74A and human HM74 amino acid sequences

FIG. 14. Alignment of human HM74 and HM74A amino acid sequences

FIG. 15. Amino acid sequence alignment of human HM74A and murine HM74A

FIG. 16. Amino acid sequence alignment of human HM74A and rat HM74A

FIG. 17. Rat and human HM74A elicit nicotinic acid responses with similar potency following expression in HEK293T cells together with Gₒ₁α

FIG. 18 Rat (A) and human (B) HM74A display similar pharmacological profiles using nicotinic acid analogues following expression in HEK293T cells together with Gₒ₁α

FIG. 19. Nucleotide (cDNA) sequence of monkey HM74A

FIG. 20. Amino acid sequence of monkey HM74A

FIG. 21. Nucleotide sequence of rabbit HM74A

FIG. 22. Amino acid sequence of rabbit HM74A

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

Prior to this invention, the utility of the HM74A and HM74 receptors was unknown. The discovery that nicotinic acid can act as a ligand indicates that diseases or conditions mediated by HM74A and/or HM74 include dyslipidemia including associated diabetic dyslipidemia and mixed dyslipidemia,), heart failure, hypercholesteremia, cardiovascular disease including atherosclerosis, arteriosclerosis, and hypertriglyceridemia, type II diabetes mellitus, type I diabetes, insulin resistance, hyperlipidemia, anorexia nervosa, obesity. It is also believed that the HM74 and HM74A receptors are involved in inflammation. Inflammation represents a group of vascular, cellular and neurological responses to trauma. Inflammation can be characterised as the movement of inflammatory cells such as monocytes, neutrophils and granulocytes into the tissues. This is usually associated with reduced endothelial barrier function and oedema into the tissues. Inflammation with regards to disease typically is referred to as chronic inflammation and can last up to a lifetime. Such chronic inflammation may manifest itself through disease symptoms. The aim of anti-inflammatory therapy is therefore to reduce this chronic inflammation and allow for the physiological process of healing and tissue repair to progress. Examples of inflammatory diseases or conditions include those of the joint, particularly arthritis (e.g. rheumatoid arthritis, osteoarthritis, prosthetic joint failure), or the gastrointestinal tract (e.g. ulcerative colitis, Crohn's disease, and other inflammatory bowel and gastrointestinal diseases, gastritis and mucosal inflammation resulting from infection, the enteropathy provoked by non-steroidal anti-inflammatory drugs), of the lung (e.g. adult respiratory distress syndrome, asthma, cystic fibrosis, or chronic obstructive pulmonary disease), of the heart (e.g. myocarditis), of nervous tissue (e.g. multiple sclerosis), of the pancreas, (e.g., inflammation associated with diabetes melitus and complications thereof, of the kidney (e.g. glomerulonephritis), of the skin (e.g. dermatitis, psoriasis, eczema, urticaria, burn injury), of the eye (e.g. glaucoma) as well as of transplanted organs (e.g. rejection) and multi-organ diseases (e.g. systemic lupus erythematosis, sepsis) and inflammatory sequelae of viral or bacterial infections and inflammatory conditions associated with atherosclerosis and following hypoxic or ischaemic insults (with or without reperfusion), for example in the brain or in ischaemic heart disease. By the term HM74 and/or HM74A mediated diseases it is meant those diseases where the modulation of HM74 and/or HM74A, by ligands other than nicotinic acid, results in a beneficial modifiction of the disease state.

HM74A and HM74 may therefore be used as screening targets for the identification and development of novel pharmaceutical agents for use in the methods of the invention. A modulator of HM74A and/or HM74 may be identified by contacting a test agent with a cell expressing on the surface the receptor HM74 and/or HM74A, said receptor being associated with a second component capable of providing a detectable signal in response to the binding of an agent to said receptor, with an agent to be screened under conditions to permit binding to the receptor; and determining whether the agent binds to, and activates, or inhibits, the receptor, by detecting the presence or absence of a signal generated from the interaction of the compound with the receptor and thereby determining whether the test agent modulates HM74A and/or HM74 activity. This may be carried out in the presence of a labelled or unlabelled ligand, e.g. nicotinic acid.

For example a method for identification of an agent that modulates HM74A and/or HM74 activity comprises:

(i) contacting a test agent with a cell, such as an adipocyte, which expresses HM74A and/or HM74 or a variant thereof which is capable of coupling to a G-protein; and

(ii) monitoring for HM74A and/or HM74 activity in the presence of a G-protein; thereby determining whether the test agent modulates HM74A and/or HM74 activity.

The test agent may be contacted in step (i) with cells that express HM74A and/or HM74 or a variant thereof. Alternatively, the test agent may be contacted in step (i) with membrane obtained from such cells.

A modulator of HM74 and/or HM74A may be identified by determining the inhibition of binding of a ligand to cells which have the receptor on the surface thereof, or to cell membranes containing the receptor, in the presence of a candidate compound, under conditions to permit binding to the receptor, and determining the amount of ligand bound to the receptor, such that a compound capable of causing reduction of binding of a ligand is an agonist or antagonist, in which method the ligand is nicotinic acid.

The invention also provides:

a test kit suitable for identification of an agent that modulates HM74A and/or HM74 activity, which kit comprises:

(a) HM74A and/or HM74 or a variant thereof which is capable of coupling to a G-protein; and

(b) means for monitoring HM74A and/or HM74 activity.

a method for identification of an agent that inhibits lipolysis, which method comprises contacting adipocytes in vitro with a test agent which modulates HM74A and/or HM74 activity and which has been identified by the method of the invention and monitoring lipolysis, thereby determining whether the test substance is an inhibitor of lipolysis;

an activator of HM74A and/or HM74 activity or an inhibitor of lipolysis identified by a method of the invention, their use in therapy and pharmaceutical compositions comprising them.

an activator of HM74A and/or HM74 activity or an inhibitor of lipolysis identified by a method of the invention or a polynucleotide which encodes HM74A and/or HM74 or a variant polypeptide, for use in a method of treatment of the human or animal body by therapy; and

use of such an activator, inhibitor or polynucleotide in the manufacture of a medicament for the treatment of diseases or conditions modulated by HM74 and/or HM74A, for example dyslipidaemia and conditions associated with dyslipidaemia, coronary heart disease, atheroselerosis, thrombosis or obesity, angina, chronic renal failure, peripheral vascular disease, stroke, type II diabetes metabolic syndrome (syndrome X) or inflammation.

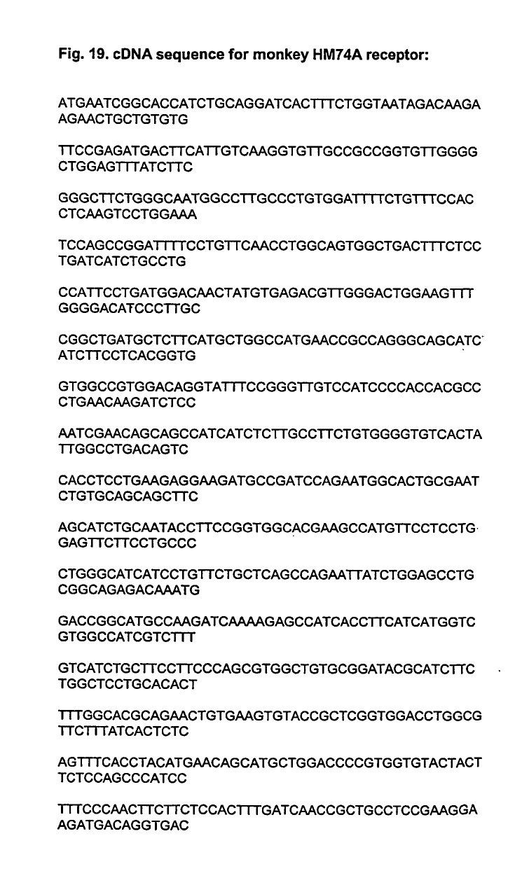

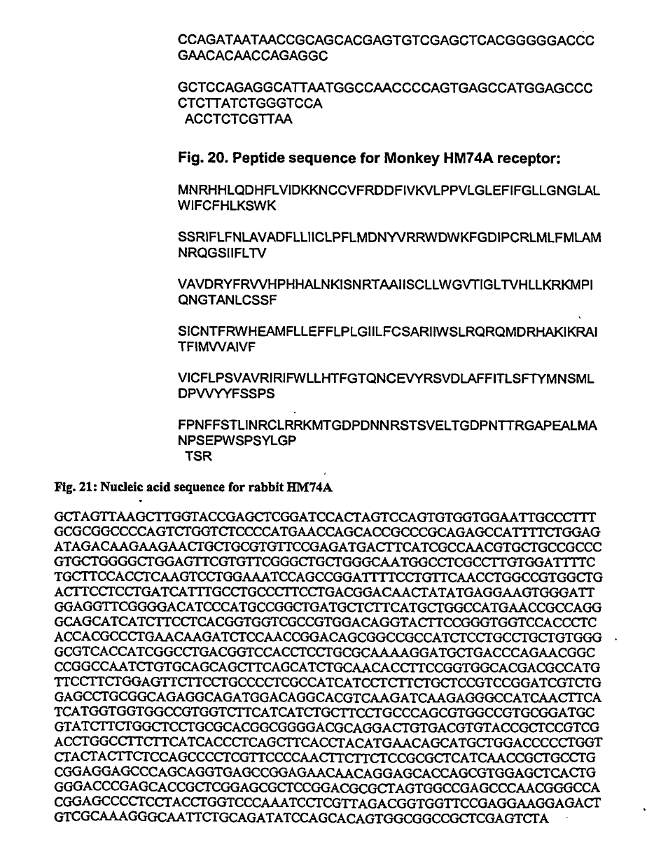

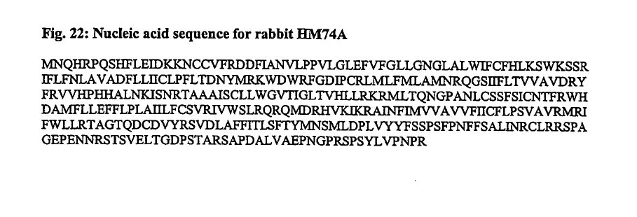

The present invention relates to the use of human G-protein coupled receptors, HM74 and HM74A, and variants thereof. HM74 has been cloned previously (Nomura, H., Nielsen, B. W. and Matsushima, K. Int. Immunol. 5: 1239-1249, (1993)). HM74 receptor has the GenBank Accession number D10923. The variant of HM74 known as HM74A is also a preferred variant. This is described below and also in WO9856820. The terms HM74A and HM74 as used herein incorporate variants of HM74 and HM74A. HM74A and HM74 receptors for use in the screening methods of the invention include all species orthologues, eg, may be rodent, mouse, rabbit, monkey or human. Rabbit and monkey nucleic acid and amino acid sequences are depicted in FIGS. 19-22. Human is especially preferred.

The term “variant” refers to a polypeptide which has the same essential character or basic biological functionality as HM74A and/or HM74. The essential characters of HM74A and HM74 can be defined as that of G-protein coupled receptors. HM74A and HM74 couple to Gᵢ G-protein. Thus, the term “variant” refers in particular to a polypeptide which activates Gᵢ.

To determine whether a candidate variant has the same function as HM74A and/or HM74, the ability of the variant to activate Gᵢ-protein can be determined. The effect of the candidate variant on Gᵢ activation can be monitored. This can be carried out, for example, by contacting cells expressing the candidate variant with a ligand which activates Gᵢ-protein when contacted with cells that express HM74A and/or HM74, and measuring a Gᵢ-coupled readout. A control experiment is typically also carried out in which cells of the same type as those expressing the candidate variant, but expressing HM74A and/or HM74 instead, are contacted with the ligand and a corresponding Gᵢ-coupled readout is measured. The effect attained by the candidate variant can then be directly compared with that attained by HM74A and/or HM74.

Alternatively, a variant polypeptide is one which binds to the same ligand as HM74A and/or HM74. That can be determined directly by contacting a candidate variant with a radiolabelled ligand that binds to HM74A and/or HM74 and monitoring binding of the ligand to the variant. Typically, the radiolabelled ligand can be incubated with cell membranes containing the candidate variant. The membranes can then be separated from non-bound ligand and dissolved in scintillation fluid to allow the radioactivity of the membranes to be determined by scintillation counting. Non-specific binding of the candidate variant may also be determined by repeating the experiment in the presence of a saturating concentration of non-radioactive ligand. Preferably a binding curve is constructed by repeating the experiment with various concentrations of the candidate variant. The ability to bind a ligand of HM74A and/or HM74 may also be determined indirectly as described below.

Typically, polypeptides with more than about 65% identity, preferably at least 80% or at least 90% and particularly preferably at least 95%, at least 97% or at least 99% identity, with the amino acid sequence of HM74 as published (Nomura et al Int. Immuno 5 1239-1249, 1993) or HM74A as described in WO9856820 or more preferably over a region of at least 20, preferably at least 30, at least 40, at least 60 or at least 100 contiguous amino acids or over the full length of the amino acid sequences are considered as HM74A and HM74 variants. The UWGCG Package provides the BESTFIT program which can be used to calculate identity (for example used on its default settings) (Devereau et al (1984) Nucleic Acid Research 12, p387-395). The PILEUP and BLAST algorithms can be used to calculate identity or line up sequences (typically on their default settings), for example as described in Algschul S. F. (1993) J. Mol. Evol. 36: 290-300; Altschul, S. F. et al (1990) J. Mol. Biol. 215: 403-10. Software for performing BLAST analyses is publicly available through the National Centre for Biotechnology Information (http://www.ncbi.nlm.nih.gov/).

Variant polypeptides therefore include naturally occurring allelic variants. An allelic variant will generally be of human or non-human mammal origin, such as bovine or porcine origin. Alternatively, a variant polypeptide can be a non-naturally occurring sequence. A non-naturally occurring variant may thus be a modified version of HM74A and/or HM74.

The amino acid sequence of HM74A and/or HM74 may be modified by deletion and/or substitution and/or addition of single amino acids or groups of amino acids as long as the modified polypeptide retains the capability to function as a G-protein coupled receptor. Such amino acid changes may occur in one, two or more of the intracellular domains of HM74A and/or HM74 and/or one, two or more of the extracellular domains of HM74A and/or HM74 and/or one, two or more of the transmembrane domains of HM74A and/or HM74.

Amino acid substitutions may thus be made, for example from 1, 2, 3, 4 or 5 to 10, 20 or 30 substitutions. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

A variant polypeptide may be a shorter polypeptide. For example, a polypeptide of at least 20 amino acids or up to 50, 60, 70, 80, 100 or 150 amino acids in length may constitute a variant polypeptide as long as it demonstrates the functionality of HM74A and/or HM74. A variant polypeptide may therefore lack one, two or more intracellular domains and/or one, two or more extracellular domains and/or one, two or more transmembrane domains. A variant polypeptide may thus be a fragment of the full length polypeptide. A shortened polypeptide may comprise a ligand-binding region (N-terminal extracellular domain) and/or an effector binding region (C-terminal intracellular domain). Such fragments can be used to construct chimeric receptors preferably with another 7-transmembrane G-coupled receptor.

Variant polypeptides include polypeptides that are chemically modified, e.g. post-translationally modified. For example, such variant polypeptides may be glycosylated or comprise modified amino acid residues. They may also be modified by the addition of histidine residues, for example 6 or 8 His residues, or an epitope tag, for example a T7, HA, myc or flag tag, to assist their purification or detection. They may be modified by the addition of a signal sequence to promote insertion into the cell membrane.

The invention also utilises nucleotide sequences that encode HM74A and/or HM74 or variants thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Such nucleotides can be isolated from human cells or synthesised according to methods well known in the art, as described by way of example in Sambrook et al, Molecular Cloning: A Laboratory Manual, 2nd edition, Cold Spring Harbour Laboratory Press, 1989. Typically a useful polynucleotide comprises a contiguous sequence of nucleotides which is capable of hybridising under selective conditions to the coding sequence or the complement of the coding sequences of HM74A and/or HM74.

A polynucleotide can hydridize to the coding sequence or the complement of the coding sequences of HM74A and/or HM74 at a level significantly above background. Background hybridisation may occur, for example, because of other cDNAs present in a cDNA library. The signal level generated by the interaction between a polynucleotide and the coding sequence or complement of the coding sequence of HM74A and/or HM74 is typically at least 10 fold, preferably at least 100 fold, as intense as interactions between other polynucleotides and the coding sequence of HM74A and/or HM74. The intensity of interaction may be measured, for example, by radiolabelling the probe, e.g. with ³²P. Selective hybridisation may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.03M sodium chloride and 0.003M sodium citrate at about 60° C.).

The coding sequences of HM74A and/or HM74 may be modified by one or more nucleotide substitutions, for example from 1, 2, 3, 4 or 5 to 10, 25, 50 or 100 substitutions. The polynucleotides of HM74A and/or HM74 may alternatively or additionally be modified by one or more insertions and/or deletions and/or by an extension at either or both ends. The modified polynucleotides generally encode polypeptides which have G-protein coupled receptor activity or inhibit the activity of HM74A and/or HM74. Degenerate substitutions may be made and/or substitutions may be made which would result in a conservative amino acid substitution when the modified sequences are translated, for example as shown in the Table above.

A nucleotide sequence which is capable of selectively hybridising to the complement of the DNA coding sequences of HM74 and HM74A will generally have at least 60%, at least 70%, at least 80%, at least 90%, at least 95%, at least 98% or at least 99% sequence identity to the coding sequence of HM74 and HM74A over a region of at least 20, preferably at least 30, for instance at least 40, at least 60, more preferably at least 100 contiguous nucleotides or most preferably over the full length. Methods of measuring nucleic acid and protein homology are well known in the art. For example the UWGCG Package provides the BESTFIT program which can be used to calculate homology (Devereux et al 1984). Similarly the PILEUP and BLAST algorithms can be used to line up sequences (for example are described in Altschul 1993, and Altschul et al 1990). Many different settings are possible for such programs. In accordance with the invention, the default settings may be used.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

Polynucleotides may be used as a primer, eg a PCR primer or a primer for an alternative amplification reaction of a probe, eg labelled with a revealing label by conventional means for identifying mutations in HM74A and/or HM74 that may be implicated in diseases resulting from abnormal lipolysis. Fragments of polynucleotides may be fused to the coding sequence of other proteins, preferably other G-protein coupled receptors, to form a sequence coding for a fusion protein.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500 nucleotides in length, or even up to a few nucleotides, such as five or ten nucleotides, short of the coding sequences of HM74A and/or HM74.

The polynucleotides have utility in production of HM74A and/or HM74 or variant polypeptides, which may take place in vitro, in vivo or ex vivo. The polynucleotides may be used as therapeutic agents in their own right, in gene therapy techniques. The polynucleotides are cloned into expression vectors for these purposes. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to a person skilled in the art. By way of further example in this regard we refer to Sambrook et al.

Expression vectors comprise a polynucleotide encoding the desired polypeptide operably linked to a control sequence which is capable of providing for the expression of the coding sequence by a host cell. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be plasmid, virus or phage vectors provided with a origin of replication, optionally a promoter for the expression of the said polynucleotide and optionally a regulator of the promoter. The vectors may contain one or more selectable marker genes, for example an ampicillin resistence gene in the case of a bacterial plasmid or a resistance gene for a fungal vector. Vectors may be used in vitro, for example for the production of RNA or DNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmtl and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters, in particular adipose cell specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide which comprise sequences homologous to eukaryotic genomic sequences, preferably mammalian genomic sequences, or viral genomic sequences. This will allow the introduction of the relevant polynucleotides into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Retrovirus vectors for example may be used to stably integrate the polynucleotide into the host genome. Replication-defective adenovirus vectors by contrast remain episomal and therefore allow transient expression.

Cells are transformed or transfected with the vectors to express the HM74A and/or HM74 polypeptides or a variant thereof. Such cells may be eucaryotic or prokaryotic. They include transient or, preferably, stable higher eukaryotic cell lines such as mammalian cells or insect cells, lower eukaryotic cells such as yeast, and prokaryotic cells such as bacterial cells. Particular examples of cells which may be used to express HM74A and/or HM74 or a variant polypeptide include mammalian HEK293T, CHO, HeLa and COS7 cells. Preferably the cell line selected will be one which is not only stable, but also allows for mature glycosylation and cell surface expression of HM74A and/or HM74 polypeptides or a variant. Cells such as adipocytes expressing HM74A and/or HM74 receptors or a variant polypeptide may be used in screening assays. Expression may be achieved in transformed oocytes. The HM74A and/or HM74 polypeptides or a variant may be expressed in cells such as adipose tissue of a transgenic non-human animal, preferably a rodent such as a mouse.

The present invention is concerned in particular with the use of HM74A and/or HM74 or a functional variant in screening methods to identify agents that may act as modulators of HM74A and/or HM74 receptor activity and, in particular, agents that may act as modulators of lipolysis. Such modulators are useful in the treatment of dyslipidaemia, coronary artery disease, atherosclerosis, obesity and thrombosis, angina, chronic renal failure, peripheral vascular disease, stroke, type II diabetes, inflammation and metabolic syndrome (syndrome X).

Any suitable form of assay may be employed to identify a modulator of HM74A and/or HM74 activity and/or of lipolysis. In general terms, such screening methods involve contacting HM74A and/or HM74 or a variant polypeptide with a test compound and then determining receptor activity. G-protein activation, and especially Gᵢ-protein activation, may be determined therefore. Where a test compound affects receptor activity, its effect on lipolysis can be determined by contacting adipocytes in culture with the test compound and measuring lipolysis.

Modulator activity can be determined in vitro or in vivo by contacting cells expressing HM74A and/or HM74 or a variant polypeptide with an agent under test and by monitoring the effect mediated by HM74A and/or HM74 or variant polypeptide. Thus, a test agent may be contacted with isolated cells which express HM74A and/or M74 or a variant polypeptide. The cells may be provided in culture. Cells may be disrupted and cell membranes isolated and used.

The HM74A and/or HM74 or variant polypeptide may be naturally or recombinantly expressed. Preferably, an assay is carried out in vitro using cells expressing recombinant polypeptide or using membranes from such cells. Suitable eucaryotic and procaryotic cells are discussed above. Preferably adipocytes are used.

Typically, receptor activity is monitored by measuring a Gᵢ-coupled readout. Gᵢ-coupled readout can be monitored using an electrophysiological method to determine the activity of G-protein regulated Ca²⁺ or K⁺ channels or by using fluorescent dye to measure changes in intracellular Ca²⁺ levels. Other methods that can typically be used to monitor receptor activity involved measuring levels of or activity of GTP(S or cAMP.

Yeast assays may be used to screen for agents that modulate the activity of HM74A and/or HM74 or variant polypeptides. A typical yeast assay involves heterologously expressing HM74A and/or HM74 or a variant polypeptide in a modified yeast strain containing multiple reporter genes, typically FUS1-HIS3 and FUS1-lacZ, each linked to an endogenous MAPK cascade-based signal transduction pathway. This pathway is normally linked to pheromone receptors, but can be coupled to foreign receptors by replacement of the yeast G protein with yeast/mammalian G protein chimeras. Strains may also contain further gene deletions, such as deletions of SST2 and FAR1, to potentiate the assay. Ligand activation of the heterologous receptor can be monitored for example either as cell growth in the absence of histidine or with a suitable substrate such as beta-galactosidase (lacZ).

Alternatively melanophore assays may be used to screen for activators of HM74A and/or HM74. HM74A and/or HM74 or a variant polypeptide can be heterologously expressed in Xenopus laevis melanophores and their activation can be measured by either melanosome dispersion or aggregation. Basically, melanosome dispersion is promoted by activation of adenylate cyclase or phospholipase C, i.e. Gₛ and G_{q} mediated signalling respectively, whereas aggregation results from activation of Gᵢ-protein resulting in inhibition of adenylate cyclase. Hence, ligand activation of the heterologous receptor can be measured simply by measuring the change in light transmittance through the cells or by imaging the cell response.

Preferably, control experiments are carried out on cells which do not express HM74A and/or HM74 or a variant polypeptide to establish whether the observed responses are the result of activation of the HM74A and/or HM74 or the variant polypeptide.

Suitable test substances which can be tested in the above assays include combinatorial libraries, defined chemical entities, peptide and peptide mimetics, oligonucleotides and natural product libraries, such as display (e.g. phage display libraries) and antibody products. In a preferred embodiment, the test substance is a nicotinic acid (Niacin). Assays may also be carried out using known ligands of other G-protein coupled receptors to identify ligands which act as agonists at HM74A and/or HM74.

Test substances may be used in an initial screen of, for example, 10 substances per reaction, and the substances of these batches which show inhibition or activation tested individually. Test substances may be used at a concentration of from 1 nM to 1000 μM, preferably from 1 μM to 100 μM, more preferably from 1 μM to 10 μM.

Agents which modulate HM74A and/or HM74 activity and which have been identified by assays in accordance with the invention can be used in the treatment or prophylaxis of lipid disorders which are responsive to regulation of HM74A and/or HM74 receptor activity. Agents which activate HM74A and/or HM74 receptor activity and/or which have been identified as inhibitors of lipolysis are preferred. In particular, such agents may be used in the treatment of dyslipidaemia and conditions associated with dyslipidaemia such as atherosclerosis, obesity, thrombosis or coronary artery disease, angina, chronic renal failure, peripheral vascular disease, stroke, type II diabetes metabolic syndrome (syndrome X) and inflammation.

The amount of a HM74A and/or M74 modulator which is required to achieve the desired biological effect will, of course, depend on a number of factors, for example, the mode of administration and the precise clinical condition of the recipient. In general, the daily dose will be in the range of 0.1 mg-1 g/kg, typically 0.1-100 mg/kg. An intravenous dose may, for example, be in the range of 0.01 mg to 0.1 g/kg, typically 0.01 μg to 10 mg/kg, which may conveniently be administered as an infusion of from 0.1 g to 1 mg, per minute. Infusion fluids suitable for this purpose may contain, for example, from 0.01 μg to 0.1 mg, per millilitre. Unit doses may contain, for example, from 0.01 μg to 1 g of a HM74 modulator. Thus ampoules for injection may contain, for example, from 0.01 μg to 0.1 g and orally administrable unit dose formulations, such as tablets or capsules, may contain, for example, from 0.1 mg to 1 g.

A HM74A and/or HM74 modulator may be employed in the treatment of a HM74A and/or HM74 mediated disease as the compound per se, but is preferably presented with an acceptable carrier in the form of a pharmaceutical formulation. The carrier must, of course, be acceptable in the sense of being compatible with the other ingredients of the formulation and must not be deleterious to the recipient. The carrier may be a solid or a liquid, or both, and is preferably formulated with the HM74A and/or HM74 modulator as a unit-dose formulation, for example, a tablet, which may contain from 0.05% to 95% by weight of the HM74 modulator.

The formulations include those suitable for oral, rectal, topical, buccal (e.g. sub-lingual) and parenteral (e.g. subcutaneous, intramuscular, intradermal or intravenous) administration.

Formulations suitable for oral administration may be presented in discrete units, such as capsules, cachets, lozenges or tablets, each containing a predetermined amount of a HM74A and/or HM74 modulator, as a powder or granules; as a solution or a suspension in an aqueous or non-aqueous liquid; or as an oil-in-water or water-in-oil emulsion. In general, the formulations are prepared by uniformly and intimately admixing the active HM74A and/or HM74 modulator with a liquid or finely divided solid carrier, or both, and then, if necessary, shaping the product. For example, a tablet may be prepared by compressing or moulding a powder or granules of the HM74A and/or HM74 modulator optionally with one or more accessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Moulded tablets may be made by moulding, in a suitable machine, the powdered compound moistened with an inert liquid diluent.

Formulations suitable for buccal (sub-lingual) administration include lozenges comprising a HM74A and/or HM74 modulator in a flavoured base, usually sucrose and acacia or tragacanth, and pastilles comprising the HM74A and/or HM74 modulator in an inert base such as gelatin and glycerin or sucrose and acacia.

Formulations of the present invention suitable for parenteral administration conveniently comprise sterile aqueous preparations of an HM74A and/or HM74 modulator, preferably isotonic with the blood of the intended recipient. These preparations are preferably administered intravenously, although administration may also be effected by means of subcutaneous, intramuscular, or intradermal injection. Such preparations may conveniently be prepared by admixing the HM74A and/or HM74 modulator with water and rendering the resulting solution sterile and isotonic with the blood. Injectable compositions according to the invention will generally contain from 0.1 to 5% w/w of the HM74A and/or HM74 modulator.

Formulations suitable for rectal administration are preferably presented as unit-dose suppositories. These may be prepared by admixing a HM74A and/or HM74 modulator with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include vaseline, lanolin, polyethylene glycols, alcohols, and combinations of two or more thereof. The HM74A and/or HM74 modulator is generally present at a concentration of from 0.1 to 15% w/w of the composition, for example, from 0.5 to 2%.

Alternatively agents which up-regulate HM74A and/or HM74 expression or nucleic acids encoding HM74A and/or HM74 or a variant polypeptide may be administered to the mammal. Nucleic acid, such as RNA or DNA, preferably DNA, is provided in the form of a vector, which may be expressed in the cells of a human or other mammal under treatment. Preferably such up-regulation or expression following nucleic acid administration will enhance HM74A and/or HM74 activity.

Nucleic acid encoding HM74A and/or HM74 or variant polypeptide may be administered to a human or other mammal by any available technique. For example, the nucleic acid may be introduced by injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to the mucosal surfaces for example by intranasal, oral, intravaginal, intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 μg to 1 mg, preferably to 1 μg to 10 μg nucleic acid for particle mediated gene delivery and 10 μg to 1 mg for other routes.

Polynucleotides encoding HM74A and/or HM74 or a variant polypeptide can also be used to identify mutation(s) in HM74 or HM74A genes which may be implicated in human disorders. Identification of such mutation(s) may be used to assist in diagnosis of dyslipidaema and conditions associated with dyslipidaemia such as, atherosclerosis, obesity, thrombosis, angina, chronic renal failure, peripheral vascular disease, stroke, type II diabetes, inflammation and metabolic syndrome (syndrome X) or other disorders or susceptibility to such disorders and in assessing the physiology of such disorders.

Antibodies (either polyclonal or preferably monoclonal antibodies, chimeric, single chain, Fab fragments) which are specific for the HM74A and HM74 polypeptides or a variant thereof can be generated. Such antibodies may for example be useful in purification, isolation or screening methods involving immunoprecipitation techniques and may be used as tools to elucidate further the function of HM74A and HM74 or a variant thereof, or indeed as therapeutic agents in their own right. Such antibodies may be used to block ligand binding to the receptors. A variety of protocols for competitive binding or immunoradiometric assays to determine the specific binding capability of an antibody are well known in the art (see for example Maddox et al, J. Exp. Med. 158, 1211 et seq, 1993).

The activators, inhibitors, polynucleotides and antibodies for use in the instant invention may be used in combination with one or more other therapeutic agents for example, 3-hydroxy-3-methylglutaryl-coenzyme A reductase inhibitors (statins), PPAR modulators (e.g. fibrates) bile acid binding resins, MTP inhibitors and LDL up-regulators. The invention thus provides in a further aspect the use of a combination of a HM74 and/or HM74A modulator (excluding nicotinic acid) and at least one other therapeutic agent, preferably a statin in the treatment of HM74 and/or HM74A mediated disorders.

When the activators, inhibitors and polynucleotides and antibodies are used in combination with other therapeutic agents, the agents may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further aspect of the invention. The individual components of such combinations may be administered either sequentially or simultaneously in separate or combined pharmaceutical formulations.

When combined in the same formulation it will be appreciated that the two components must be stable and compatible with each other and the other components of the formulation and may be formulated for administration. When formulated separately they may be provided in any convenient formulation, conveniently in such a manner as are known for such compounds in the art.

When in combination with a second therapeutic agent active against the same disease, the dose of each component may differ from that when the compound is used alone. Appropriate doses will be readily appreciated by those skilled in the art.

The present invention will now be further illustrated by the accompanying Examples which do not limit the scope of the invention in any way.

### EXAMPLE 1

To identify possible candidate nicotinic acid receptors, Taqman RNA data was investigated to shortlist 11 orphan receptors which were expressed in adipose and/or omentum together with spleen (Table 1).

TABLE 1
Orphan GPCRs, which by Taqman mRNA analysis share adipose
and/or omental fat and spleen tissue distribution

| | Tissue distribution | | |
|---|---|---|---|
| Orphan GPCR | Adipose | Omentum | Spleen |
| P2Y9 | + | + | − |
| P2Y5 | + | + | − |
| GPR5 | + | − | + |
| GPR52 | + | − | + |
| EBI2 | − | + | + |
| GPR34 | − | + | + |
| HM74 | + | + | + |
| DezB | + | − | + |
| GPR15 | + | − | + |
| HIPHUM 10 | + | + | + |
| HIPHUM 15 (EDG8) | − | + | − |

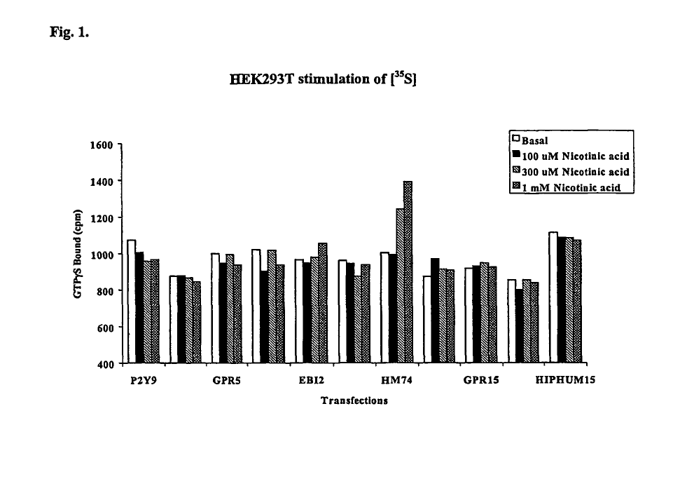

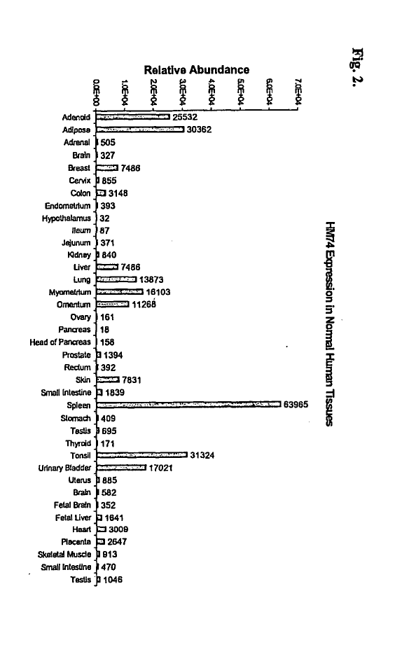

Each of the cDNAs for these orphans were then transiently transfected into HEK293T cells together with that of the Gᵢ family G protein Gₒ₁α and responses measured-using [³⁵S]GTPγS binding following exposure of membranes to nicotinic acid. FIG. 1 shows that only membranes from cells transfected to express the orphan receptor HM74 yielded responses to nicotinic acid. No such responses were measured from any other transfected membranes. Taqman mRNA distribution analysis is depicted in FIG. 2. HM74 mRNA was found to be highly expressed in those tissues commensurate with a nicotinic acid receptor ie spleen, subcutaneous adipose and omental fat.

### EXAMPLE 2

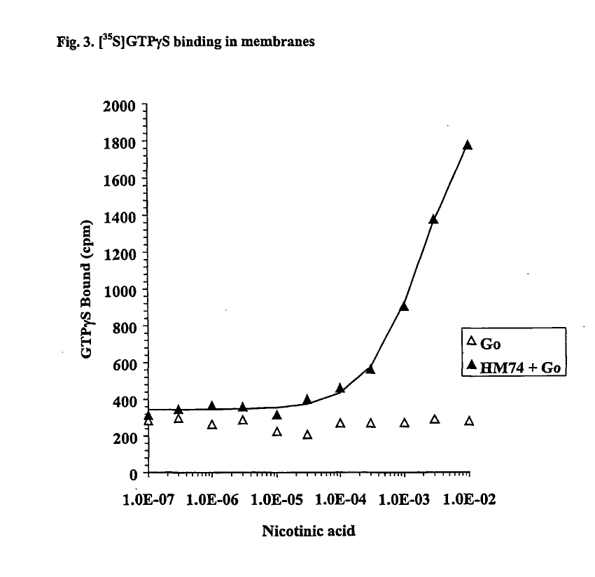

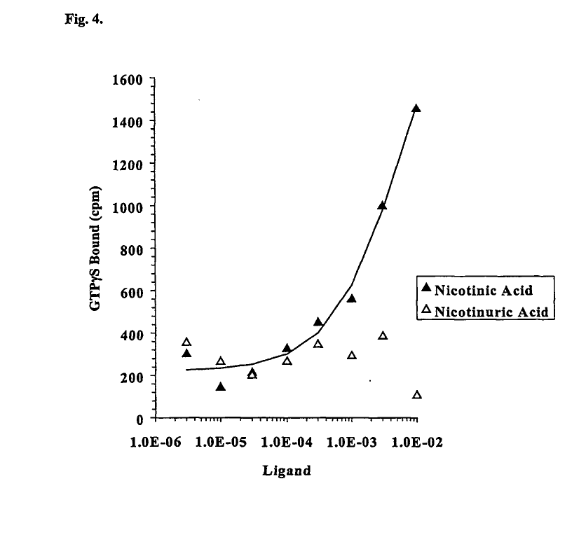

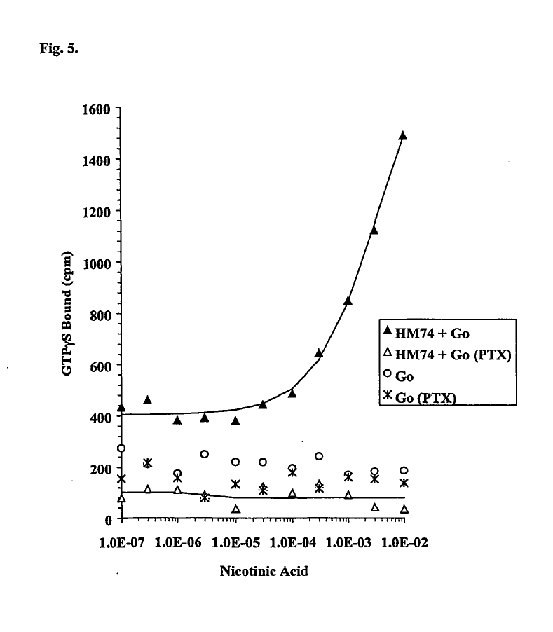

Further detailed functional analysis of HM74 showed that nicotinic acid elicited a robust, concentration-dependent stimulation of [³⁵S]GTPγS binding on membranes from HM74-expressing HEK293T cells (FIG. 3). In addition, nicotinuric acid, a structurally related metabolite of nicotinic acid, was not found to alter basal [³⁵S]GTPγS binding in such membranes (FIG. 4). The α subunits of the Gᵢ-like G protein family bind and hydrolyse guanine nucleotides at significantly higher rates than other G protein families such as G_{q}, Gₛ and G₁₂. Therefore, ligand stimulation of [³⁵S]GTPγS binding is usually indicative of receptor-mediated activation of Gᵢ G proteins. With the exception of G_{z}α, the α subunits of the Gᵢ-like G proteins all possess a conserved cysteine residue four amino acids from the C-terminus which acts as an acceptor for ADP-ribosylation catalysed by pertussis toxin (3). The addition of the ADP-ribose group serves to prevent GPCR-Gᵢ coupling. Therefore, a simple means of defining whether a cellular response to an agonist at a GPCR is transduced via activation of a Gᵢ-like G protein is to record attenuation of function following pertussis toxin treatment of cells. FIG. 5 shows that pertussis toxin pre-treatment of HM74 and Gₒ₁α-expressing HEK293T cells (50 ng/ml, 16 h) completely abolished nicotinic acid-mediated stimulation of [³⁵S]GTPγS binding. These data provide strong evidence that nicotinic acid promotes activation of Gᵢ G protein signalling via HM74.

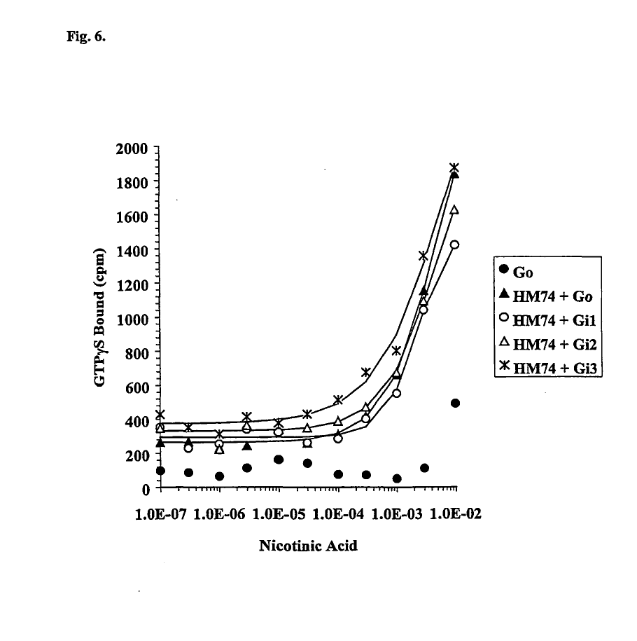

To investigate whether HM74 displayed selectivity for any particular Gᵢ-like G protein, HEK293T cells were co-transfected with HM74 cDNA in combination with cDNAs for Gₒ₁α, Gᵢ₁α, Gᵢ₂α and Gᵢ₃α. The ability of various concentrations of nicotinic acid to stimulate [³⁵S]GTPγS binding was measured in membranes from transfected cells. FIG. 6 shows that similar concentration-response curves and maximal stimulations were achieved following co-expression of HM74 with each of the Gᵢ-like G proteins. These data suggest that the nicotinic acid-responsive HM74 receptor has the capacity to interact with all four G₁-like G proteins and to promote their activation with similar efficiency. The potency of the nicotinic acid response at recombinant HM74 is at least 100-1000-fold right-shifted compared to that measured in native ratadipose and/or spleen which maybe due to species receptor variation.

### EXAMPLE 3

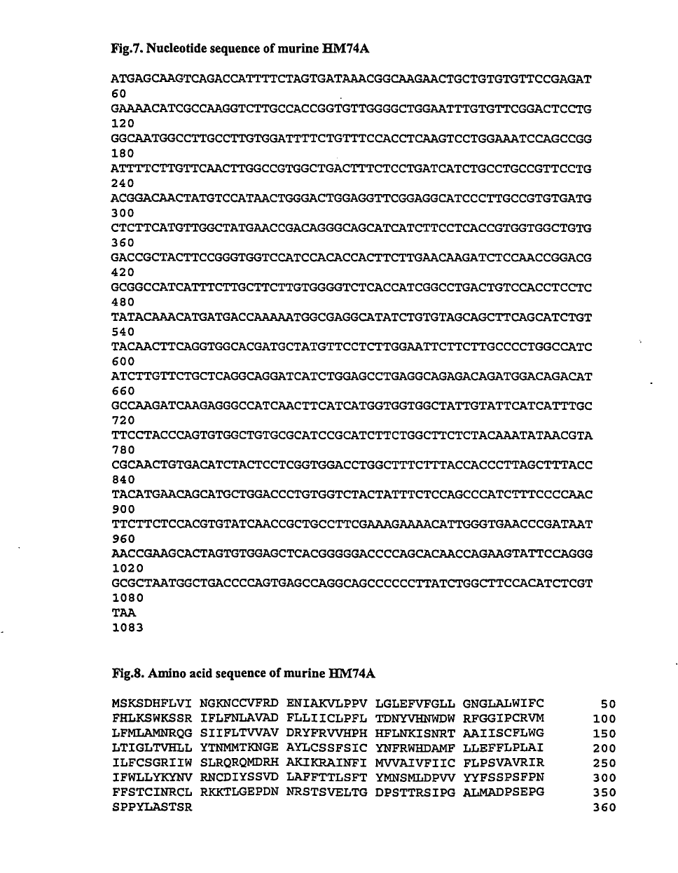

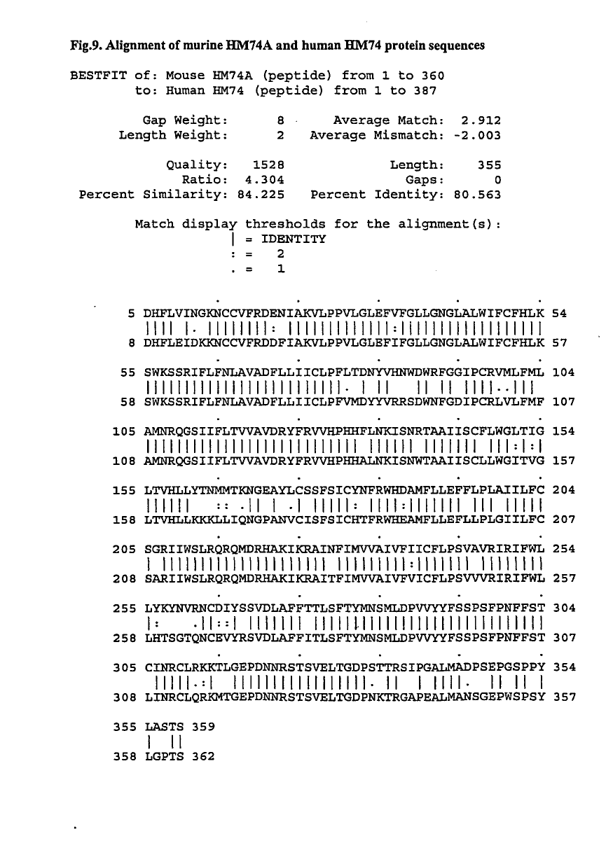

In order to identify species orthologues of HM74, a murine sequence with significant homology to human HM74 was identified by searching public domain databases with the peptide sequence for human HM74 taken from GenBank entry Accession No. D10923. A TBLASTN search produced significant alignments with Accession No.s AJ300198 and AJ300199 describing the Mus musculus Puma-g gene for a putative seven transmembrane spanning receptor (termed HM74A). Noticeably, the encoded peptide sequence had a shorter C-terminal tail when compared to that of the human gene. BESTFIT analysis (Wisconsin Package Version 10.1, Genetics Computer Group (GCG)) showed approx. 81% identity/84% similarity across the region of ungapped alignment (see FIGS. 7-9).

Using the murine sequence information, a set of 4 PCR primers were designed to try and amplify regions from the 5′ and 3′ ends of the corresponding rat gene.

Primer NF454 (5′-ACTGGCCAGATCCACTCATG-3′) is identical to the sequence immediately 5′ to and including the putative start codon of the murine gene (AJ300198/AJ300199) and was used with primer NF450 (5′-GAACGGCAGGCAGATGATCAG-3′) whose sequence is identical to conserved corresponding regions of the coding sequence of D10923. (human HM74) and AJ300198/AJ300199 (murine HM74A). These primers were used in combination to amplify a fragment of approx. 250 bp from rat genomic DNA corresponding to the 5′ end of the gene.

This region was amplified using Pfu DNA polymerase under conditions recommended by the manufacturer (Stratagene) using an annealing temperature of 50° C., cloned into the vector PCR-Script and sequenced. 3 clones were found to have identical sequence and a new PCR primer (NF459 5′-TAGGATCCACCATGAGCAAGCAGAACCACTT-3′) was designed based on the sequence including and immediately 3′ to the putative start codon (underlined in bold). Additional sequence 5′ to the start codon was included to encode a BamHI restriction site and a Kozak sequence (believed to help optimise translation).

A 370 bp fragment of DNA corresponding to the 3′ end of the rat gene was amplified using primers NF455 (5′-ACATGAACAGCATGCTGGAC-3′) whose sequence is identical to conserved corresponding regions within the coding sequence of D10923 and AJ300198/AJ300199 and NF458 (5′-GATTCTCCGAATCTAGAAGTTCCA-3′) whose sequence is identical to a region 3′ to the putative stop codon in AJ300198/AJ300199 and has 21/24 bases in common with a region 3′ to the stop codon in D10923.

This region was amplified using Pfu DNA polymerase under conditions recommended by the manufacturer (Stratagene) using an annealing temperature of 50° C., cloned into the vector PCR-Script and sequenced. 3 clones were found to have identical sequence. Sequence analysis suggested that the putative stop codon in the rat was in a position analogous to that in the mouse, rather than being further 3′ as in the human.

A new PCR primer (NF460 5′-TACTCGAGTTAACGAGATGTGGAAGCCA-3′) was designed based on the sequence including and immediately 5′ to the putative stop codon. Additional sequence 3′ to the stop codon was included to encode a XhoI restriction site.

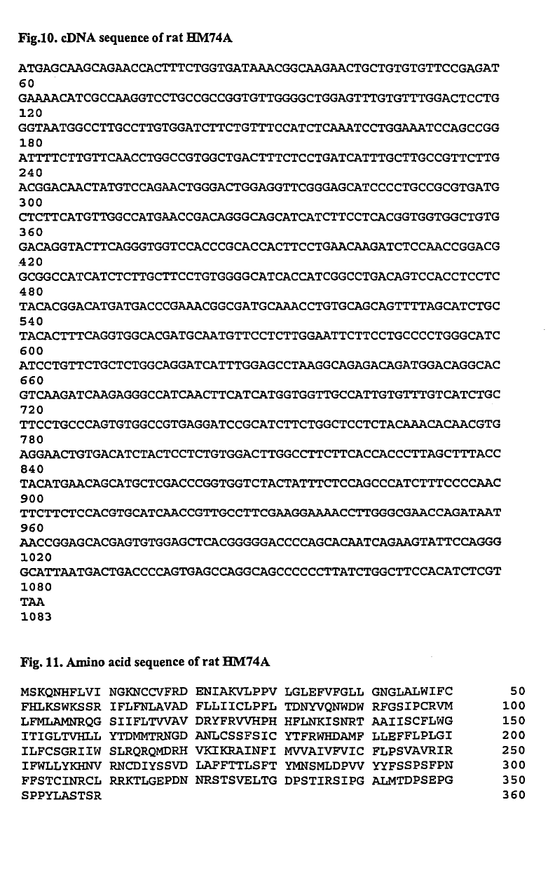

Primers NF459 and NF460 were used to amplify a fragment containing the entire coding sequence of the rat gene. This region was amplified using Pfu DNA polymerase under conditions recommended by the manufacturer (Stratagene) using an annealing temperature of 55° C., cloned into the vector PCR-Script and sequenced. 3 clones were found to have identical sequence. Full cDNA and translated amino acid sequences are shown in FIGS. 10 and 11, respectively.

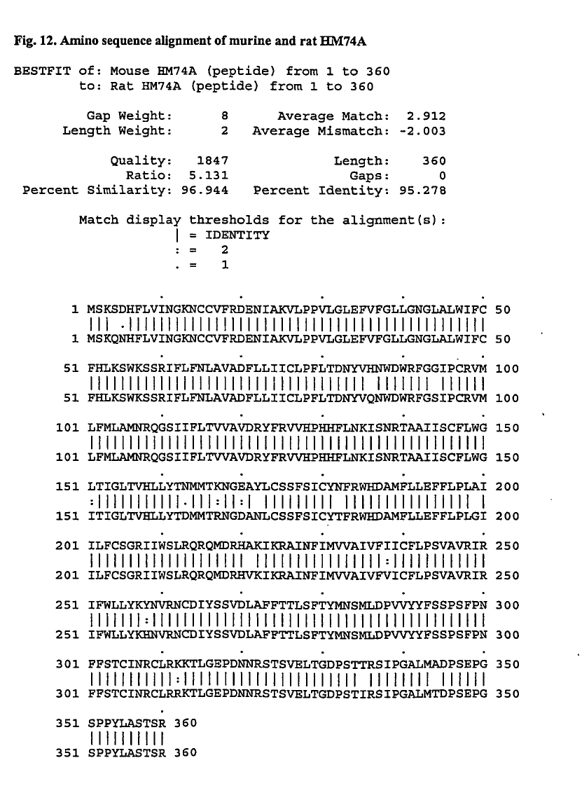

A comparison of the rat and mouse sequences using BESTFIT analysis (Wisconsin Package Version 10.1, Genetics Computer Group (GCG)) showed approx. 95% identity/97% similarity (FIG. 12).

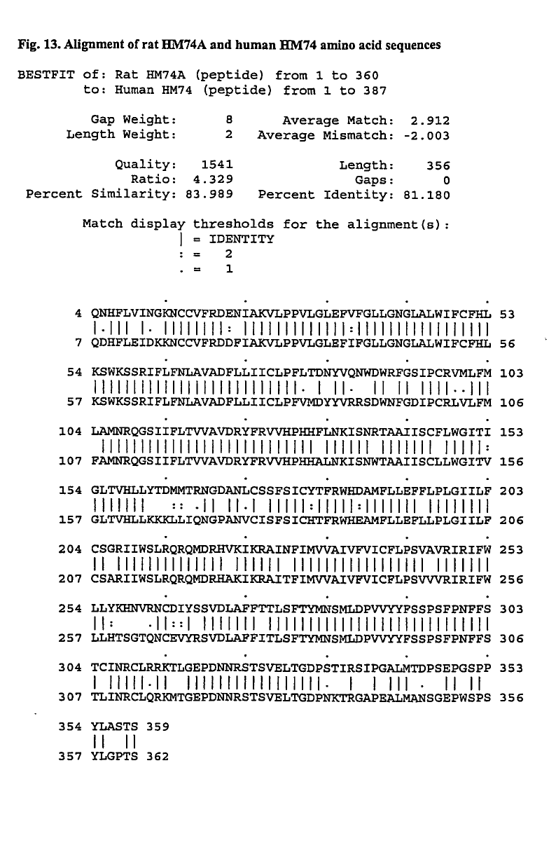

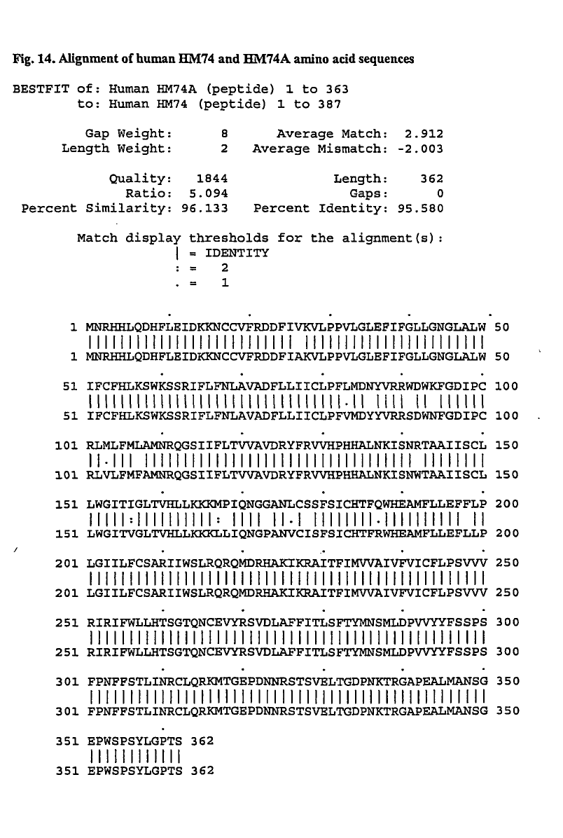

A comparison of the rat and human sequences showed approx. 81% identity/84% similarity (FIG. 13).

A TBLASTN search of the public domain databases using the rat peptide sequence indicated that there were two regions similar to this in the high throughput draft sequence for homo sapiens chromosome 12 clones RP11-507N20 and RP11-324E6 as represented by GenBank Accession No.s AC026331 and AC026333 respectively. One of these regions represents the same sequence as that present in GenBank Accession No. D10923 (human HM74). The other represents a sequence which shares approximately 96% identity/96% similarity across the region of ungapped alignment to HM74 (FIG. 14) and which we term HM74A. HM74A would also be similar in length to the mouse and rat sequences that have been described herein.

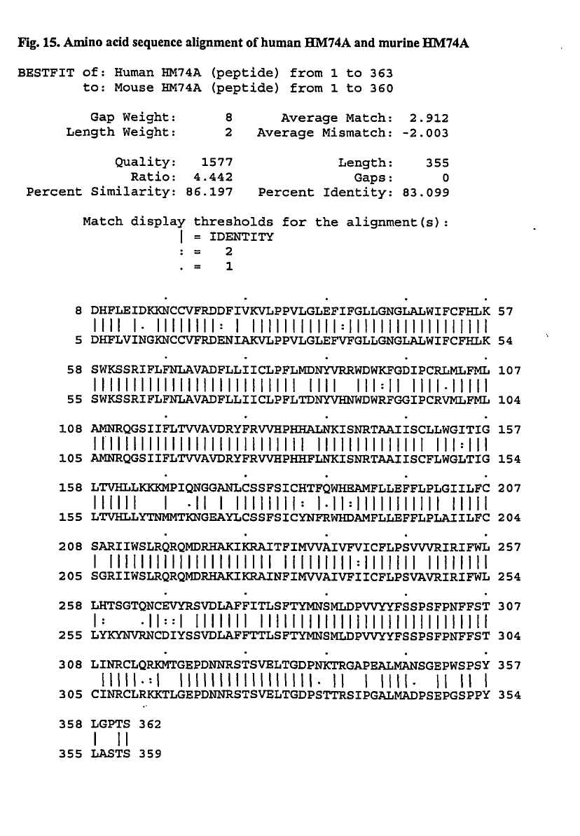

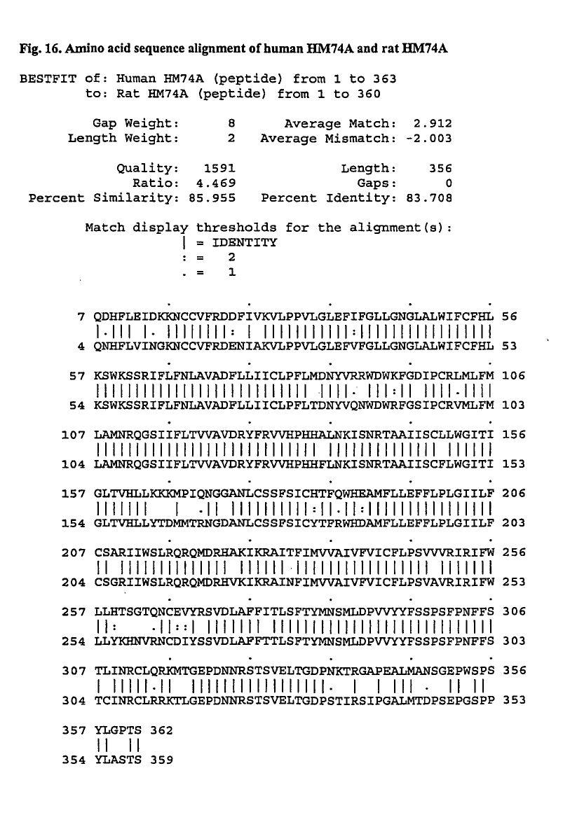

Human HM74A is 83% identical/86% similar to the mouse gene represented by Accession No.s AJ300198 and AJ300199 (FIG. 15) and is 84% identical/86% similar to the rat sequence whose isolation is described here (FIG. 16).

The mouse and rat sequences described here are likely to be the species orthologues of HM74A rather than HM74. HM74 appears to be part of a duplication of a section of human chromosome 12, or its equivalent, in the relatively recent evolutionary past.

### EXAMPLE 4

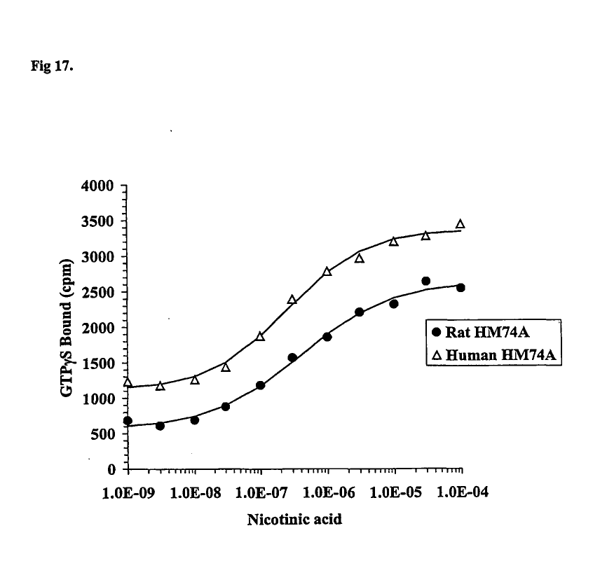

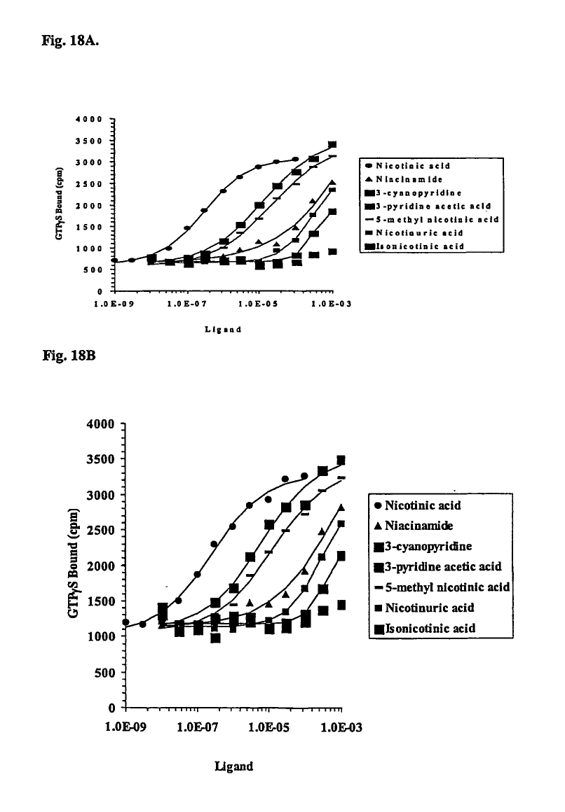

Rat HM74A was transfected into HEK293T cells in combination with Gₒ₁α and GTPγS binding measured on membranes in response to nicotinic acid exposure. Parallel experiments were performed using human HM74A. FIG. 17. shows that nicotinic acid stimulated GTPγS binding in both rat and human HM74A-transfected membranes in a concentration-dependent manner with an approx. EC₅₀ of 400 nM. This value is very similar to that measured in native adipose and spleen tissue and suggests that HM74A represents a high affinity nicotinic acid receptor. A more detailed pharmacological characterisation was then carried out on rat and human HM74A using a number of nicotinic acid analogues. FIG. 18. shows that rat (A) and human (B) HM74A display very similar pharmacological profiles with identical rank orders of potency and which are also in good agreement with published data on rat adipose and spleen tissue.

### EXAMPLE 5

Methods

Mammalian Cell Culture and Transfections

HEK293T cells (HEK293 cells stably expressing the SV40 large T-antigen) were maintained in DMEM containing 10% (v/v) foetal calf serum and 2 mM glutamine. Cells were seeded in 60 mm culture dishes and grown to 60-80% confluency (18-24 h) prior to transfection with pCDNA3 containing the relevant DNA species using Lipofectamine reagent. For transfection, 3 μg of DNA was mixed with 10 μl of Lipofectamine in 0.2 ml of Opti-MEM (Life Technologies Inc.) and was incubated at room temperature for 30 min prior to the addition of 1.6 ml of Opti-MEM. Cells were exposed to the Lipofectamine/DNA mixture for 5 h and 2 ml of 20% (v/v) newborn calf serum in DMEM was then added. Cells were harvested 48-72 h after transfection.

Preparation of Membranes

Plasma membrane-containing P2 particulate fractions were prepared from cell pastes frozen at −80° C. after harvest. All procedures were carried out at 4° C. Cell pellets were resuspended in 1 ml of 10 mM Tris-HCl and 0.1 mM EDTA, pH 7.5 (buffer A) and by homogenisation for 20 s with a polytron homogeniser followed by passage (5 times) through a 25-guage needle. Cell lysates were centrifuged at 1,000 g for 10 min in a microcentrige to pellet the nuclei and unbroken cells and P2 particulate fractions were recovered by microcentrifugation at 16,000 g for 30 min. P2 particulate fractions were resuspended in buffer A and stored at −80° C. until required. Protein concentrations were determined using the bicinchoninic acid (BCA) procedure (4) using BSA as a standard.

High Affinity [³⁵SGTPγS Binding.

Assays were performed in 96-well format using a method modified from Wieland and Jakobs, 1994 (5). Membranes (10 μg per point) were diluted to 0.083 mg/ml in assay buffer (20 mM HEPES, 100 mM NaCl, 10 mM MgCl₂, pH7.4) supplemented with saponin (10 mg/l) and pre—incubated with 40 μM GDP. Various concentrations of nicotinic acid were added, followed by [³⁵S]GTPγS (1170 Ci/mmol, Amersham) at 0.3 nM (total vol. of 100 μl) and binding was allowed to proceed at room temperature for 30 min. Non-specific binding was determined by the inclusion of 0.6 mM GTP. Wheatgerm agglutinin SPA beads (Amersham) (0.5 mg) in 25 μl assay buffer were added and the whole was incubated at room temperature for 30 min with agitation. Plates were centrifuged at 1500 g for 5 min and bound [³⁵S]GTPγS was determined by scintillation counting on a Wallac 1450 microbeta Trilux scintillation counter.

Assays for Compound Screening

Modulator activity can be determined by contacting cells expressing a polypeptide of the invention with a substance under investigation and by monitoring the effect mediated by the polypeptides. The cells expressing the polypeptide may be in vitro or in vivo. The polypeptide of the invention may be naturally or recombinantly expressed. Preferably, the assay is carried out in vitro using cells expressing recombinant polypeptide. Typically, receptor activity can be monitored indirectly by measuring a Gᵢ-coupled readout. Gᵢ coupled readout can typically be monitored using an electrophysiological method to determine the activity of G-protein regulated Ca²⁺ or K⁺ channels or by using a fluorescent dye to measure changed in intracellular Ca²⁺ levels. Other methods that can typically be used to monitor receptor activity involve measuring levels of or activity of GTPγS or cAMP.

Preferably, control experiments are carried out on cells which do not express the polypeptide of the invention to establish whether the observed responses are the result of activation of the polypeptide.

Mammalian cells, such as HEK293, CHO and COS7 cells over-expressing the protein of choice are generated for use in the assay. Cell lines which maybe employed as suitable hosts include i) CHO cells transfected to stably express PLC 2, a PLC isoform which allows Gᵢ G proteins to elicit Ca²⁺ mobilisation or ii) CHO cells transfected to stably express the G_{q} family G protein G₁₆ together with a suitable reporter gene e.g. NFAT (nuclear factor activator of T cells). Expression of G₁₆ permits a wide variety of non-G_{q} coupled receptors to mobilise Ca²⁺.

96 and 384 well plate, high throughput screens (HTS) are employed using a) fluorescence based calcium indicator molecules, including but not limited to dyes such as Fura-2, Fura-Red, Fluo 3 and Fluo 4 (Molecular Probes); or b) reporter gene read-out Secondary screening involves the same technology. A brief screening assay protocol is as follows:—

Mammalian cells stably over-expressing the protein are cultured in black wall, clear bottom, tissue culture coated 96 or 384 well plates with a volume of 1001 μl cell culture medium in each well 3 days before use in a FLIPR (Fluorescence Imaging Plate Reader—Molecular Devices). Cells were incubated with 4 μM FLUO-3AM at 30° C. in 5% CO₂ for 90 mins and then washed once in Tyrodes buffer containing 3 mM probenecid. Basal fluorescence was determined prior to compound additions. Activation results in an increase in intracellular calcium which can be measured directly in the FLIPR.

The binding of a modulator to a polypeptide of the invention can also be determined directly. For example, a radio labeled test substance can be incubated with the polypeptide of the invention and binding of the test substance to the polypeptide can be monitored. Typically, the radiolabeled test substance can be incubated with cell membranes containing the polypeptide until equilibrium is reached. The membranes can then be separated from a non-bound test substance and dissolved in scintillation fluid to allow the radioactive content to be determined by scintillation counting. Non-specific binding of the test substance may also be determined by repeating the experiment in the presence of a saturating concentration of a non-radioactive ligand.

Yeast Assays

Polypeptides can be heterologously expressed in a modified yeast strain containing multiple reporter genes, typically FUS1-HIS3 and FUS1-lacZ, each linked to an endogenous MAPK cascade-based signal transduction pathway. This pathway is normally linked to pheromone receptors, but can be coupled to foreign receptors by replacement of the yeast G protein with yeast/mammalian G protein chimeras. Strains also contain two further gene deletions, of SST2 and FAR1, to potentiate the assay. Ligand activation of the heterologous receptor can be monitored either as cell growth in the absence of histidine or with a substrate of beta-galactosidase (lacZ). This technology is described in WO99/14344.

Melanophore Assays

Polypeptide of the invention can be heterologously expressed in Xenopus laevis melanophores and its activation can be measured by either melanosome dispersion or aggregation Basically, melanosome dispersion is promoted by activation of adenylate cyclase or phospholipase C ie Gs and Gq mediated signalling, respectively, whereas aggregation results from activation of Gi G proteins resulting in inhibition of adenylate cyclase. Hence, ligand activation of the HM74 can be measured simply by measuring the change in light transmittance through the cells or by imaging the cell response.

1. A method for identifying an agents which modulates the activity of an HM74A and/or HM74 receptor which comprises determining whether the agent interacts with HM74A and/or HM74.

2. A method according to claim 1 wherein the method is carried out in combination with a ligand for HM74A and/or HM74.

3. A method according to claim 2 wherein the ligand is nicotinic acid.

4. A method of identifying a modulator of HM74A and/or HM74 comprising contacting an agent with a cell expressing on the surface the receptor HM74 and/or HM74A, said receptor being associated with a second component capable of providing a detectable signal in response to the binding of an agent to said receptor, with an agent to be screened under conditions to permit binding to the receptor; and determining whether the agent binds to, and activates, or inhibits, the receptor, by detecting the presence or absence of a single generated from the interaction of the agent with the receptor and thereby determines whether the agent modulates HM74A and/or HM74 activity.

5. A method according to claim 1 wherein the receptor is selected from human, rodent, murine, rabbit and monkey receptors.

6. A method according to claim 5 wherein the receptor is the rodent, human or murine receptor.

7. A method according to claim 6 wherein the receptor is the human receptor.

8. A method according claim 1 wherein the cells are adipocytes.

9. A method according to claim 8 wherein the adipocytes are provided as a differentiated cell line.

10. A method according to claim 8 wherein the adipocytes are primary adipocytes harvested from a human or animal donor.

11. A method according to claim 4 wherein the second component capable of providing a detectable signal is a G-protein.

12. A method according to claim 11 wherein the G-protein is Gᵢ-protein.

13. A test kit suitable for identification of an agent that modulates HM74 activity, which kit comprises:
a) HM74A or HM74 or a variant thereof which is capable of coupling to a Gᵢ-protein; and
b) means for monitoring HM74 or HM74A activity.

14. A kit according to claim 13 wherein component (a) comprises cells which express HM74 or HM74A or a said variant thereof.

15. A kit according to claim 13 wherein component (b) comprises means for determining whether Gᵢ-protein is activated.

16. A method for identification of an agent that inhibits lipolysis, comprising contacting adipocytes in vitro with an agent identified by the method of claim 1 and monitoring lipolysis, thereby determining whether the test agent is an inhibitor of lipolysis.

17. An activator of HM74A and/or HM74 activity identified by a method according to claim 1.

18. An activator according to claim 17 for use in a method of treatment of the human or animal body by therapy.

19. An activator according to claim 17 for use in the treatment of dyslipidaemia, coronary heart disease, atherosclerosis, thrombosis or obesity, angina, chronic renal failure, peripheral vascular disease, stroke, type II diabetes metabolic syndrome (syndrome X) or inflammation.

20. A medicament for the treatment of dyslipidaemia, coronary heart disease, atherosclerosis, thrombosis or obesity, angina, chronic renal failure, peripheral vascular disease, stroke, type II diabetes metabolic syndrome (syndrome X) or inflammation, comprising an activator of claim 17.

21. A medicament for the treatment of HM74A and/or HM74 mediated diseases, comprising a modulator of HM74A and/or HM74 with the proviso the modulator is not nicotinic acid.

22. A method of treating HM74A and/or HM74 mediated disease in an individual which comprises administering to the individual a therapeutic amount of a modulator of HM74A and/or HM74 activity with the proviso the compound is not nicotinic acid.

23. The method of claim 22 further comprising administering at least one other therapeutic agent in the treatment of HM74 and/or HM74A mediated disorders.

24. Use according to claim 23 wherein at least one other therapeutic agents includes a statin.

25. An inhibitor of lipolysis identified by a method according to claim 16.

26. A medicament for the treatment of dyslipidaemia, coronary heart disease, atherosclerosis, thrombosis or obesity, angina, chronic renal failure, peripheral vascular disease, stroke, type II diabetes metabolic syndrome (syndrome X) or inflammation, comprising an inhibitor of claim 25.

27. A polynucleotide which encodes HM74A and/or HM74 or a variant thereof identified by a method according to claim 1.

28. A medicament for the treatment of dyslipidaemia, coronary heart disease, atherosclerosis, thrombosis or obesity, angina, chronic renal failure, peripheral vascular disease, stroke, type II diabetes metabolic syndrome (syndrome X) or inflammation, comprising a polynucleotide of claim 27.
